(19) United States
(12) Patent Application Publication
(10) Pub. No.: US 2009/0081156 A1
(43) Pub. Date: Mar. 26, 2009

(54) Bioactive molecular matrix and methods of use in the treatment of disease

(76) Inventor: William Soo Hoo, Carlsbad, CA (US)

Correspondence Address: WESLEY B. AMES, 7031 LOS VIENTOS SERENOS, ESCONDIDO, CA, 92029, US

(21) Appl. No.: 11/888,520

(22) Filed: Aug. 1, 2007

Related U.S. Application Data
(60) Provisional application No. 60/835,599, filed on Aug. 3, 2006.

(51) Int. Cl. A61K 39/39; A61K 38/20; A61K 38/21; A61K 39/00; A61K 39/12

(52) U.S. Cl. 424/85.2; 424/278.1; 424/282.1; 424/130.1; 424/85.5; 424/85.7; 424/184.1; 424/204.1

(57) ABSTRACT

The present invention provides methods and compositions for stimulating an immune response or modulating cell signal transduction in a host by administering to said host a composition comprising at least three biomodulatory molecules connected by at least one cross-linking agent forming a chain or matrix wherein the chain or matrix functions as an immuno-stimulatory adjuvant to activate an immune accessory cell.

The composition may comprise one or more types of biomodulatory molecules selected from the group consisting of cytokines, bacterial molecules, receptor ligands, antigen binding fragments of antibodies, heat shock proteins, and integrins. The composition may further comprise one or more disease-specific antigens to stimulate an immune response. The disease-specific antigens may be selected from the group consisting of tumor-associated antigens, infectious disease-associated antigens, autoimmune-associated antigens, parasitic antigens, bacterial antigens, and viral antigens. In addition the composition may further comprise a solid support to which the cross-linked biomodulatory molecules are affixed. The solid support may be selected from the group consisting of Dextran, chitosan, alginate, poly-DL lactide polyglycolide, polyglycolide, or alum.

### CROSS-REFERENCES TO RELATED APPLICATIONS

This application is a continuation-in-part of patent application Ser. No. 60/835,599 filed 3 Aug. 2006.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Sequence listing is provided on pages 56 through 70.

### BACKGROUND OF THE INVENTION

1. Field of the Invention

The present invention relates generally to immunology and molecular biology, more specifically the use of a bioactive molecular matrix to effect an immune response in human or veterinary applications.

2. Description of Related Art

In the fight against cancer and infectious diseases, vaccines have required the use of immune stimulating compounds known as adjuvants. Unfortunately, in the nearly 70 year history of vaccines, the only adjuvant approved for use in man are salts of aluminum hydroxide (Alum™). The resulting immune responses to tumors have been negligible and immunity to infectious diseases have been limited. A variety of cancer immunotherapies are known in the art to elicit, enhance or boost an immune response including those that utilize microorganisms known to stimulate a non-specific local immune response, biological response modifiers such as cytokines and interferons, monoclonal antibodies directed against particular tumor antigens, tumor cells to provide tumor antigen, tumor cell extracts which provide higher concentrations of tumor antigens, tumor cell extracts in conjunction with cytokines and cancer cells that have been transformed to express a membrane bound immunomodulatory fusion protein.

A variety of microorganisms or fractions of microbial products such as C. parvum, Bacille, S. typhimurium, M. tuberculosis, and BCG cell walls are known to elicit a wide range of host responses that activate neutrophils, macrophages, NK cells, T cells, and B cells and their products, many of which can mediate tumor-cell killing. Unfortunately, the mechanism of tumor cell killing appears to be an “innocent bystander” effect mediated by a vigorous local immune response and little if any boosting of systemic reactions. Although systemic immunity has been detected in some experiments, there has been little success in developing a sustained potent immune response sufficient to reduce tumor size. In addition, it is difficult to relate the conditions for successful experimental immunotherapy in animals to the clinical circumstance in man since local control of human cancer is rarely an issue in view of surgical resection and radiation therapy techniques. Rather the more crucial issue is effective treatment of metastatic disease, a setting for which an elicitation of a local, innate immune response would be largely insufficient. Finally, while tumors have been killed as an innocent bystander of a granulomatous inflammatory response, systemic immunity is rarely elicited and systemic toxicity is seen and sometimes fatal. Furthermore none of these approaches to the treatment of cancer has produced long term disease free survival of patients with metastatic disease.

Biological response modifiers mediate a wide range of biological responses. For example, a class of cytokines known as interferons elicit biological responses such as anti-viral effects, antiproliferative effects, cytotoxic effects, inhibition of angiogenesis, immunomodulation, gene activation, and differentiation. Because of these effects interferons have been found useful against a number of infectious and immune disorders and is a treatment of choice for some cancers. Unfortunately, the ideal dosage for each patient is difficult to determine and application of the inappropriate dosage can have significant detrimental effects. Furthermore, in a physiologic setting, cytokine molecules are relatively concentrated in the location where they are needed and are transient in expression. This has made clinical use of purified cytokines difficult. For example, if the primary goal of interferon treatment is to effect tumor proliferation the maximal tolerated dose is preferable, however, if the treatment is to maximally boost the immune response against the tumor a lower optimal immunomodulatory dose would be preferable. An effective dosage will depend on the potency of the molecule administered as well as the availability of molecule to interact with receptors. For example, clinical use of soluble interferon, TNF-α, and IL-2 have met with limited success due to the fact that the concentrations necessary to have effects at the tumor site result in a concomitant rise in systemic toxicity (Taguchi, T. and Sohumura, Y.; Biotherapy 3:177, 1991). Because current protocols administer these molecules in solution a higher dose is generally required to effect a target cell, consequently continued exposure of nearby cells to these dosage concentrations often cause toxic effects. This toxicity is manifested as fatigue, weakness, anorexia, weight loss, fever, and lethargy. Correspondingly, at lower dosages most of the effects are not easily assessed or monitored in the patient, consequently, treatment dosages are difficult to determine and may not be effective. As in the case of TNF-α, injection of the soluble form results in significant toxicity. In another example, high doses of soluble IL-2 actually resulted in the inability to induce an anti-tumor response in the BALB/c mouse tumor model. Other attempts at using whole cell vaccines genetically modified to secrete soluble cytokines are still undergoing testing in the clinic but have demonstrated some albeit limited success. Although these strategies use tumor cells to supply tumor-associated antigens for immune recognition, the low success rate may be due to the lack of a specific response modifier that is integrally linked to the antigen source. As with all ex vivo cell therapies, isolation, modification, and characterization of individual patient cells is time consuming, costly, and presents numerous manufacturing and regulatory problems. In addition, the amounts of cytokines secreted by tumor cells vary greatly, making dosing difficult. Furthermore, the secretion of soluble molecules, which may lessen the amount of systemic toxicity, fails to address the problem of unwanted free molecules diffusing to detrimentally affect other tissues. Also, the diffusion of free bioactive molecules reduces the amount of available molecules to bind specific ligands in a localized area where they are needed.

The earliest development of antibodies against human tumors was conducted using antibodies coupled to another, more toxic reagent to fashion a “magic bullet” that would specifically seek out tumor cells and destroy them. A large variety of cytotoxic agents have been described in the art. The most commonly used are radioisotopes and chemical toxins. Chemical toxins include for example protein toxins, cytotoxins, and chemotherapeutic agents. However, each of these coupled reagents has its own unique disadvantages as well as common disadvantages associated with the antibody targeting vehicle. Antibody-coupled radioisotopes have the disadvantage of irradiating adjacent tissues even in the absence of specific antibody binding. Consequently healthy tissue may be damaged or destroyed with this type of treatment. Chemical toxins have a similar disadvantage. Many chemical toxins are plant or bacterial products that are extremely toxic at doses of only a few molecules per cell and can bind directly to the cell surface without antibody coupling resulting in the damage or destruction of healthy tissue. Common disadvantages associated with the antibody targeting vehicles includes a host immune responses directed against foreign antibodies, in particular against the Fc region and to a lesser extent antibody Fab′₂ fragments. These responses can seriously compromise a cancer patient consequently, treatment with foreign monoclonal antibodies is not preferable and development of humanized antibodies currently used in the treatment of cancer requires a significant commitment of resources making this strategy less attractive.

The principle of vaccination or immunization utilizing tumor cells as an antigen source to elicit an immune response has been pursued for many years in connection with cancer. These treatments have included the administration of both unmodified and modified tumor cells. Unmodified cells include autologous or allogeneic tumor cells while modified tumor cells are cells that have been inactivated by a number of methods including radiation, freeze-thawing, heat, or chemical treatment. Unfortunately, administration of tumor cells or even inactivated tumor cells has generally proven ineffective in the elicitation of systemic immune responses against tumors.

Specific attempts at immunotherapy utilized immunization with tumor cells or tumor cell extracts either alone or in vaccines, often in conjunction with immune stimulators such as BCG have been almost uniformly unsuccessful in man and have largely been abandoned. The difficulties in eliciting an immune response with tumor cells and BCG may be due to the method in which the tumor cells and BCG have previously been displayed. Procedures have generally involved administration of a mixture of BCG and tumor cells in solution or encapsulating both within a porous matrix such as alum, microspheres, micelles, or liposomes allowing each to “leak” through the pores (U.S. Pat. No. 6,193,970). This strategy has not resulted in potent systemic immunity. The simultaneous presentation of tumor cell antigen and BCG in sufficient quantity to initiate a response often requires the administration of a high dosage of both the stimulation molecule and tumor antigen to allow sufficient interaction with receptors. Other attempts at using whole cell vaccines genetically modified to secrete soluble cytokines are still undergoing testing in the clinic but have demonstrated some albeit limited success. Although these strategies use tumor cells to supply tumor-associated antigens for immune recognition, the low success rate may be due in part to the lack of a specific immune response modifier that is integrally linked to the antigen source.

Recent interest in dendritic cell biology have made these cells attractive mediators for the immunotherapy of cancer. One strategy has been to remove dendritic cells from the body, induce maturation and pulse them with antigen. These dendritic cells are then injected into the patient. For example, administration of dendritic cells from mice have been pulsed in vitro with antigen then reinfused into the body (Inaba K et al., J. Exp. Med. 1990; 172:631). However most dendritic cells do not survive more than two days when injected (Josien R et al., J. Exp. Med. 2000; 191:495). To increase cell survival, dendritic cells have been further manipulated ex vivo such as by treatment with CD40L and TRANCE prior to injection (Josien R et al., J. Exp. Med. 2000; 191:495). However, this technique is labor intensive requiring removal and manipulation of dendritic cells prior to administration.

Consequently, there is a need in the field for a treatment that evokes an effect that specifically attacks and destroys or inactivates tumor cells leaving healthy cells unaffected, does not have significant toxicity associated with administration and is able to boost the host natural immune response against tumor cells, including metastatic tumor cells which lead to new tumor formation.

### BRIEF SUMMARY OF THE INVENTION

In one aspect of the present invention a composition is provided comprising at least three biomodulatory molecules, connected by at least one cross-linking agent forming a matrix wherein the matrix functions as an immuno-stimulatory adjuvant to activate immune accessory cells (e.g., dendritic cells, NK cells, macrophages, B cells). One aspect of the present invention targets dendritic cells in situ with biomodulatory efficacy while supplying tumor-associated (or disease-associated) antigens for efficient antigen presentation. Antigen presentation by dendritic cells is accomplished by several factors which must work in concert for efficient stimulation and subsequent immune responses. First, antigen must be present with an additional dendritic cell specific stimulus. In the case of dendritic cells GM-CSF or other appropriate biomodulatory molecule stimulates the maturation of dendritic cells resulting in the migration of the cells to draining lymph nodes thus initiating the immune response.

In the case of receptor-mediated stimulation, the number of receptors bound by ligands (biomodulatory molecules) is proportional to the amount of stimulation. Thus, engagement of at least three or more stimulatory receptors with the specific biomodulatory molecules will result in efficacious dendritic cell activation.

In addition, the increased avidity resulting from multiple biostimulatory molecules anchored in a solid support matrix increases the overall binding affinity of the particle with the dendritic cell maximizing the efficiency of association with antigenand stimulus.

In one embodiment of the present invention the biomodulatory molecule may be a cytokine, a bacterial molecule, a receptor ligand, a functional domain of a receptor molecule, an antigen-binding fragment of an antibody, a heat shock protein, or an integrin. When the biomodulatory molecule is a cytokine it may be GM-CSF, IL-2, IL-12, IFN-α, IFN-γ, TNF-α, or TNF-β. When the biomodulatory molecule is a bacterial toxin the toxin may be Staphylococcal enterotoxin B (SEB). When the biomodulatory molecule is a bacterial molecule it may be monophosphoryl lipid A, diphosphoryl lipid A or lipopolysaccharide. When the biomodulatory molecule is a bacterial molecule it may be a bacterial oligonucleotide such as a CpG motif. Preferably the CpG motif is 5′-TCC ATG ACG TTC CTG ATG CT-3′ (SEQ ID NO. 1) or a sequence that is 80% homologous to 5′-TCC ATG ACG TTC CTG ATG CT-3′ (SEQ ID NO. 1). Alternatively, the bacterial biomodulatory molecule may be Bacillus Calmette Guerin (BCG). When the biomodulatory molecule is a receptor ligand it may be TNF-α, and CD40L. When the biomodulatory molecule is a functional domain of a receptor, it may be ICOS (Inducible T cell Co-stimulator), CD28, CTLA-4. When the biomodulatory molecule is an antigen-binding fragment of an antibody it may be a fragment of the anti-CD40, anti-TNFR I, anti-TNFR II, anti-TLR (toll-like receptor), or anti-GMCSFR antibody. When the biomodulatory molecule is a heat shock protein it may be HSP65. When the biomodulatory molecule is an integrin the integrin may be CD54.

In another aspect the composition of the present invention may further comprise a disease-specific antigen wherein the composition is able to stimulate an immune response. The disease-specific antigen may be a tumor-associated antigen, an infectious disease-associated antigen, an autoimmune-associated antigen, a parasitic antigen, a bacterial antigen or a viral antigen. The disease specific antigen may also be a lysate from a tumor cell or a tumor cell line. When the disease-specific antigen is a tumor-associated antigen it may be a melanoma antigen, or mutants thereof, a bcr/abl breakpoint peptide, HER-2/neu or HPV. When the disease-specific antigen is a melanoma antigen it may be a MAGE-1, MAGE-2, MAGE-3, BAGE, GAGE-1 GAGE-2, MART-1 or tyrosinase antigen.

When the disease-specific antigen is a melanoma antigen it may be gp100. In the case where the antigen is non-disease specific, this invention can prove useful in eliciting responses to antigens against proteins, lipids, carbohydrates, nucleic acids, or other macromolecules to which one would desire an immune response against.

In yet another aspect the composition of the present invention may further comprise a solid support wherein the cross-linked biomodulatory molecules are affixed to the support. The solid support may be, for example, Dextran, chitosan, alginate, polyDL lactide coglycolide, polylactide-glycolide, inactivated viruses, viral capsids, large molecular weight proteins such as keyhole limpet hemocyanin (KLH), or alum.

In still another aspect of the present invention a pharmaceutical composition comprising any of the compositions above are provided.

In yet another aspect of the invention methods of treating a disease, stimulating an immune response or modulating cell signal transduction in a host by administering to the host any of the compositions above are provided. The stimulated response may be a cell mediated response or a humoral response. Innate immune responses are also relevant to this invention since the invention can be directed to interact with dendritic cells, macrophages or other cells through the appropriate identified receptors.

### BRIEF DESCRIPTION OF THE SEVERAL VIEWS OF THE DRAWINGS

Not Applicable

### DETAILED DESCRIPTION OF THE INVENTION

Prior to setting forth the invention, it may be helpful to an understanding thereof to first set forth definitions of certain terms that will be used hereinafter. All references, which have been cited below, are hereby incorporated by reference in their entirety.

“Biomodulatory molecule” as used herein refers to any biological compound that modulates the mammalian immune response including those of mammalian, yeast, or bacterial origin and may be characterized as molecules that modulate cellular metabolism, transcription, translation, or signal transduction. For example, a biomodulatory molecule may be an immunostimulatory molecule, an immunosuppressive molecule, a cytokine, a chemokine, or a bioactive fragments thereof.

“Crosslinking agent” as used herein refers to a structural compound able to covalently bind at least two molecules together such as for example disuccinimidyl suberate (DSS), Dextran, polylactide glycolide (PLA), chitosan, alginate, or alum.

“Matrix” or “bioactive molecular matrix” as used herein refers to at least three biomodulatory molecules bound together in any arrangement or formation by at least one crosslinking reagent in order to increase the number of receptor ligand interactions between the particle and the immune accessory cell.

“Immune accessory cell” as used herein refers to cells involved in the regulation of immune responses such as for example dendritic cells, macrophages, B-cells, T-cells, karotinocytes, eosinophils, neutrophils, natural killer (NK) cells, basophils, and myeloid cells.

“Immunostimulatory molecule” as used herein shall mean a biomodulatory molecule that initiates, promotes or enhances the production of an immune response.

“Immunosuppressive molecule” as used herein shall mean a biomodulatory molecule that interferes, reduces or inhibits the production of an immune response.

“Cytokine” as used herein refers to a biomodulatory molecule that is a member of a class of proteins that are produced by cells of the immune system and regulate or modulate an immune response. Such regulation or modulation may occur within the humoral or the cell mediated immune response and includes for example regulation and/or modulation of the effector function of T cells, B cells, NK cells, macrophages, antigen-presenting cells or other immune system cells playing roles in adaptive or innate immunity.

“Bioactive fragments” as used herein refers to one or more portions of a biomodulatory molecule that retains at least one biological function and at least 10% of the activity of that biological function of the biomodulatory molecule.

“Disease-associated antigen” or “disease-specific antigen” as used herein is a molecule that may become the target of an adaptive or innate immune response resulting in an interference of the disease pathology. Disease-associated antigens may be selectively expressed on particular disease cells, or may be expressed on both diseased and normal cells. These antigens may be isolated from a diseased cell, a cell that has been modified to express the antigen or an immunogenic epitope thereof, or a pathogen. Alternatively the antigen or immunogenic epitope thereof may be synthesized by techniques known to those skilled in the art.

“Immunogenic epitope thereof” as used herein in reference to a disease-associated antigen, means a portion of an antigen that functions as an antigenic determinant to induce an adaptive or innate immune response against the antigen.

“Polymeric support” or “solid support” as used herein refers to any biologically inert macromolecule having repetitive units onto which a matrix or bioactive molecular matrix may be conjugated. In one embodiment of the present invention, a solid support is provided comprising a matrix of biomodulatory molecules that may be utilized as a vaccine. The biomodulatory molecules are bound to the solid support. Antigen is either captured within or attached to the matrix or co-administered as a combined bolus or cocktail with the matrix. Administration may comprise injection of the bioactive molecular matrix intradermally such that immune accessory cells are exposed to the composition. The vaccine may be given as a single dose or multiple doses as well as single site or multiple site injections and may depend on the antigen and biomodulatory molecules used in the composition.

The bioactive molecular matrix functions by displaying one or more types of biomodulatory molecules in a bioactive molecular matrix in combination with antigen. This design allows immune accessory cell such as an immature dendritic cell to interact with at least two biomodulatory molecule thus increasing the number of receptor-ligand interactions on the matrix thereby initiating an immune stimulatory effect. Cellular receptors either transduce signals via a change in conformation which activates a cascade of intracellular molecular events resulting in the desired cellular effect (e.g., proliferation, mobilization, cytokine secretion, etc.). In the event that multiple receptors on a given cell are engaged by specific ligand, the concentration of the intracellular signals are concomitantly higher resulting in more efficient signaling and stimulation. Cellular receptors may also become internalized when engaged with ligand and migrate across the membrane to interact with intracellular molecules. Higher numbers of internalized receptor-ligand complexes will also result in more efficient cell stimulation. In the case of dendritic cell or other immune accessory cells, such stimulation will result in a more efficient initiation of the immune response. Such receptor-ligand crosslinks promote potent stimulation signals to the targeted cell. For example, dendritic cells are stimulated to uptake and process antigen provided by the matrix, and function as strong antigen presenting cells (“APCs”). The APC will quickly migrate to lymph nodes and activate T-cells against the displayed antigen thus creating an immune response against the antigen.

Difficulties previously observed in initiation of a immune response are overcome uniquely due to the multiple valence characteristics of the bioactive molecular matrix described herein. These difficulties include toxicity and lack of potency of solubilized biomodulatory molecules. Toxicity occurs because of the need to administer high concentrations of soluble biostimulatory molecules to provide a desired effect. Toxicity is overcome by the present invention because the biomodulatory molecules are crosslinked in a matrix. This design allows for administration of a lower dosage and reduces the detrimental effects of high concentrations of solubilized biomodulatory molecules.

Any biomodulatory molecule able to be crosslinked to form a matrix and able to effect a cell is applicable to the present invention. The effect may be to stimulate, suppress, recruit target cells for the purpose of activation, or function as a molecule increasing adherence to the cell. For example, molecules such as cytokines, bioactive fragments, cytokine agonists, bacterial antigens, immunostimulating oligonucleotides, ligands for cell receptors, heat shock proteins, antibodies to cell surface receptors and integrins may be utilized with the present invention. In addition, molecules that specifically bind cell bound receptors such as CD54 can be used in conjunction with biostimulatory molecules to enhance interaction of target cells with the matrix.

The biomodulatory molecule may be immunostimulatory or immunosuppressive. One example of biomodulatory molecules are cytokines. Cytokines are typically small proteins or glycoproteins having a molecular mass of less than 30 kDa. Although cytokines occasionally exhibit autocrine or endocrine activity, most act in a paracrine fashion and bind specific receptors on the membrane of target cells, thereby triggering signal transduction pathways that can alter gene expression. Cytokines generally display very high affinity for their cognate receptors, with disassociation constants ranging from about 10⁻⁹ to 10⁻¹² M. Due to this high affinity, picomolar concentrations of cytokines can mediate biological effects.

The term cytokines encompasses a variety of biomodulatory molecules including for example, cytokines secreted by lymphocytes (designated lymphokines) or cytokines secreted by monocytes and macrophages (designated monokines); interleukins, for example, interleukin-2 (IL-2), interleukin-4 (IL-4) and interleukin-12 (IL-12), which are molecules secreted by leukocytes that primarily affect the growth and differentiation of hematopoietic and immune-system cells; hematopoietic growth factors, for example, colony stimulating factors such as colony stimulating factor-1 (CSF-1), granulocyte colony stimulating factor (G-CSF) and granulocyte macrophage colony stimulating factor (GM-CSF) (The Cytokine Handbook (2^{nd} Edition) London: Harcourt, Brace & Company, (1994)).

The term cytokine, as used herein, encompasses cytokines produced by the T helper 1 (T_{H1}) and T helper 2 (T_{H2}) subsets. IL-2, IL-12, interferon-γ (IFN-γ and tumor necrosis factor-α (TNF-α and IFN-γ are cytokines produced by T_{H1} cells and are responsible for classical cell-mediated functions such as activation of cytotoxic T lymphocytes and macrophages and delayed-type hypersensitivity. T_{H1} cytokines are particularly useful in stimulating an immune response to tumor cells, infected cells, and intracellular pathogens.

Interleukin 4, 5, 6 and 10 are cytokines produced by T_{H2} cells and function effectively as helpers for B-cell activation and are particularly useful in stimulating an immune response against free living bacteria and helminthic parasites. T_{H2} cytokines also mediate allergic reactions.

Those skilled in the art would recognize that not only biomodulatory molecules but bioactive fragments of those molecules would be effective in the present invention. Such active fragments may be a polypeptide having substantially the same amino acid sequence as a portion of the biomodulatory molecule, provided that the fragment retains at least one biological function and at least 10% of the activity of that biological function of the biomodulatory molecule. Active fragments of cytokines are known in the art and include, for example, the nine amino acid peptide from IL-1α, VQGEESNDK, which retains the immunostimulatory activity of the full-length IL-1α cytokine (Hakim et al., J. Immunol. 157:5503 (1996)). Activity can be determined by a variety of well known in vitro and in vivo assays, such as for example, bone marrow proliferation assay (see Thomson, supra, 1994).

A cytokine antagonist may also be an immunosuppressive molecule useful in the present invention. Such cytokine antagonists may be naturally occurring or non-naturally occurring and include for example, antagonists of GM-CSF, G-CSF, IFN-γ, IFN-α, TNF-α, TNF-β, IL-1, IL-2, IL-3, IL-4, IL-6, IL-7, IL-10 and IL-12. Cytokine antagonists include cytokine deletion and point mutants, cytokine derived peptides, and soluble, dominant negative portions of cytokine receptors. Naturally occurring antagonists of IL-1, for example, can be used in a vaccine of the invention to inhibit the pathophysiological activities of IL-1. Such IL-1 antagonists include IL-1Ra, which is a peptide that binds the IL-1 receptor I with an affinity roughly equivalent to that of IL-1α or IL-1β but that does not activate the receptor (Fischer et al., Am. J. Physiol. 261:R442 (1991) and Dinarello and Thompson, Immunol. Today 12:404 (1991)). IL-1 antagonists also include IL-1α derived peptides and IL-1 mutiens (Palaszynski et al., Biochem. Biophys. Res. Commun. 147:204 (1987).

TABLE I
Exemplary Cytokines

| Cytokine | Reference |
|---|---|
| Interleukin-1 | Dinarello, Adv. Immunol. |
| (IL-1, IL-1α, IL-1β) | 44: 153 (1989) |
| Interleukin-2 | Devos et al., Nucl. Acids Res. |
| (IL-2) | 11: 4307 (1983) |
| Interleukin-3 | Yang et al., Cell |
| (IL-3) | 47: 3 (1986) |
| Interleukin-4 | Yakota et al., PNAS |
| (IL-4) | 83: 5894 (1986) |
| Interleukin-5 | Harada et al., Nature |
| (IL-5) | 134: 3944 (1985) |
| Interleukin-6 | Hirano, et al., Nature |
| (IL-6) | 324: 73 (1986) |
| Interleukin-7 | Goodwin et al., PNAS |
| (IL-7) | 86: 302 (1989) |
| Interleukin-9 | Yang et al., Blood |
| (IL-9) | 74: 1880 (1989) |
| Interleukin-10 | Vieira et al., PNAS |
| (IL-10) | 88: 1172 (1991) |
| Interleukin-11 | Paul et al., PNAS |
| (IL-11) | 87: 7512 (1990) |
| Interleukin-12 | Wolf et al., J. Immunol. |
| (IL-12) | 146: 3074 (1991) |
| Interleukin-13 | Cherminsi et al., J. Exp. Med. |
| (IL-13) | 166: 1229 (1987) |
| | Brown et al., J. Immunol. |
| | 142: 679 (1989) |
| Interleukin-14 | Ambrus et al., PNAS |
| (IL-14) | 90: 6330 (1993) |
| Interleukin-15 | Grabstein et al., Science |
| (IL-15) | 264: 965 (1994) |
| Interleukin-16 | Baier et al., PNAS |
| (IL-16) | 94: 5273 (1997) |
| Interferon-α | Pestka et al., Annu. Rev. Biochem. |
| (IFN-α) | 56: 727 (1987) |
| Interferon-β | Pestka et al., Supra |
| (IFN-β) | |
| Interferon-γ | Vilcek et al., Lymphokines |
| (IFN-γ) | 11: 1 (1985) |
| Leukemia-inhibitory factor | Gearing et al., Annals NY Acad. Sci. |
| (LIF) | 62: 8919 (1991) |
| Oncostatin M | Malik et al., Mol. Cell. Biol. |
| (OSM) | 9: 2847 (1989) |
| Transforming growth factor β | Sporn and Roberts (Eds.), |
| (TGF-β) | Handbook of Exp. |
| | Par. Springer-Verlag Vol. 65: 419 |
| Tumor necrosis factor-α | Wang et al., Science |
| (TNF-α) | 228: 149 (1985) |
| Tumor necrosis factor-β | Gray et al., Nature |
| (TNF-β) | 312: 721 (1984) |
| Granulocyte macrophage colony | Lee et al., PNAS |
| Stimulating factor | 82: 4360 (1984) |
| (GM-CSF) | |
| Colony stimulating factor 1 | Kawasaki et al., Science |
| (CSF-1) | 230: 291 (1985) |
| Granulocyte colony stimulating | Negata et al., Science |
| Factor | 319: 415 (1986) |
| (GCSF) | |
| Macrophage chemotactic and | Furutani et al., Biochem. Biophys. Res. |
| Activating factor | Comm. |
| (MCAF) | 159: 249-255 (1989) |
| Neutrophil-activating protein 2 | Walz et al., J. Exp. Med. |
| (NAP-2) | 170: 1745 (1989) |
| Platelet factor 4 | Poncz et al., Blood |
| (PF-4) | 69: 219 (1987) |

Preferably, cytokines utilized with the present invention include GM-CSF, G-CSF, TNF-α, TNF-related apoptosis inducing ligand (TRIAL), IL-1, IL-2, IL-3, IL-4, IL-6, IL-7, IL-10 and IL-12.

Any bacterial molecule, such as a bacterial toxin or bacterial immunostimulatory oligonucleotide, alone or in combination with at least one other molecule that modulates a cell involved in the immune response is also useful in the present invention. Some examples of bacterial toxin molecules are Staphylococcal Enterotoxin B, lipopolysaccharide (LPS), monophosphoryl lipid A, and diphosphoryl lipid A because of their stimulatory effects on dendritic cells.

Bacterial oligonucleotides comprising unmethylated cytosine-phosphorothioate-guanine (CpG) are known immune stimulatory sequences (ISS) and have been shown to stimulate dendritic cells, monocytes, NK cells, and B cells and may be utilized in the present invention (Weiner et al., J. Immunol. 165: 6244 (2000)). In addition, CpG motifs have been shown to enhance the immune response stimulated by GM-CSF (Weiner et al. Blood 92:3730 (1998)). A biomodulatory matrix comprising GM-CSF and a CpG motif may yield additional stimulation and may be desired when targeting dendritic cells. Moreover, CpG oligonucleotides have been shown to shift an immune response from a T_{H2} response to a T_{H1} response (Harding et al., J. Exp. Med. 186:1623 (1997)). The sequence 5′-TCC ATG ACT TTC CTG ATG CT-3′ (SEQ ID NO. 1) is the preferred CpG sequence with the present invention. Particular sequences useful and optimal for the stimulation of species-specific immunity are known and would be preferred with the present invention.

Any ligand that targets toll-like receptors (TLR; such as, for example, TLR-7, TLR-9) inducing activation, maturation of dendritic cells, langerhans cells, or cells of the innate immune system (natural killer cells, macrophages, for example) that ultimately results in an immune response are useful in the present invention.

Any ligand that targets an immune receptor inducing proliferation or apoptosis, leading to immune activation or immune suppression would be useful with the present invention. Some examples are GM-CSF targeting GM-CSF receptor, TNF-α targeting TNFR I and TNFR II, and CD40L. CD40L is expressed on T cells and helps induce the activation of professional antigen presenting cells such as dendritic cells. Binding to CD40 on dendritic cells causes “super activation” which help dendritic cells mature in the absence of T cell help. One skilled in the art would recognize that antibodies or antibody fragments against specific immune receptors such as GMCSF receptor would also be useful in stimulating an immune response.

Heat shock proteins (HSPs) are also immunomodulatory molecules useful in the present invention. Heat shock proteins, which are generally induced by stress causing conditions such as heat shock or glucose deprivation, can produce a generalized inflammatory response which can aid in elimination of, for example, tumor cells or infected cells. Heat shock proteins are distinguished by their molecular mass and grouped in families that include HSP110, HSP90, HSP70, HSP60, HSP25, HSP20 and HSP8.5. Several heat shock proteins, including HSP60, HSP70 and HSP90, are expressed on the cell surface of mycobacteria-infected cells, HIV-infected cells or tumor cells (Multhoff et al., Int. J. Cancer 61:1 (1985)). The mycobacterial heat shock protein HSP65 (Silva et al., Infect. Immun. 64:2400 (1996)) is an example of an immunomodulatory molecule useful in the invention.

Antibody and antibody fragments such as Fab′₂, Fab and light chain fragments against cell surface proteins that are able to stimulate dendritic cells such as CD40 may be substituted for ligands to modulate the immune response. In addition antibody or antibody fragments against proteins that function as cellular adhesion molecules such as ICAM may be used to enhance interaction between the matrix and the target cell. Antibody fragments may be desired over whole antibodies to prevent an immune response against the Fc region while allowing more accessability to surface receptors because of the smaller size of the fragment and the increased number of fragments able to be conjugated to the matrix. Preferrably, the antibody or antibody fragment conjugated to the matrix preferably retains 20% of the unconjugated antibodies activity, more preferably 50% of the activity, and most preferrably 90% of the original activity.

Integrins may be useful in the present invention to enhance interaction between the matrix and the target cell. A matrix comprising ICAM may increase the interaction at the matrix with the cell and thus facilitate receptor binding with the biomodulatory matrix. The molar amount of ICAM to be utilized in the present invention may be determined by known cellular adhesion assays.

The matrix of the present invention is constructed by crosslinking at least three biomodulatory molecules to form a three dimensional structure of a desired activity. The number of biomodulatory molecules will vary depending on the activity of the modulatory molecule and the required number of receptor interactions determined to modulate the cell. For example, the major histocompatibility complex molecule L^{d} requires about 1×10⁵ interactions to stimulate a T cell to function as an effector cell. The matrix may be tightly packed or may be formed into an open net- or web-like structure such that other molecules may be incorporated into the matrix or constructed into a shell-like structure bound to a biomodulatory molecule allowing other molecules such as antigens to be encased by the matrix. A variety of crosslinking agents are available commercially for interconnecting protein molecules (Pierce Chemical Co. Rockford, Ill.). Typical crosslinking agents may be heterobifunctional or homobifunctional and may interact with a number of chemical moieties on the surface of the protein. The moieties most often utilized for cross-linking include amine moieties, carboxyl moieties and sulfhydryl moieties. Homobifuctional crosslinking reagents include Bis-[β-(4-azidosalicylamido)ethyl]disulfide (BASED) and ethylene glycol bis[succinimidyl]succinate (EGS). Heterobifunctional crosslinking reagents include long chain succinimidyl-4-[N-maleimidomethyl]-cyclohexane-1-carboxyl-[6-amidocaproate] (LC-SMCC), long chain succinimidyl 6-[3-(2-pyridyldithio)-propionamido]hexanoate (LC-SPDP), N-[β-maleimidopropionic acid]hydrazide•TFA (“BMPH”) and N-κ-maleimidoundecanoic acid (KMUA). In addition, crosslinking agents may be trifunctional such as sulfosuccinimidyl [2-6-(biotinamido)-2-p-azidobenzamido)-hexanoamido]ethyl-1,3′-dithiopropionoate (Sulfo-SBED).

One or more types of biomodulatory molecules may be used to construct the matrix, for example, a single biomodulatory molecule such as GM-CSF may form the entire matrix or a combination of two or more biomodulatory molecules may be combined to form the matrix such as GM-CSF and IL-2. The construction of a single type biomodulatory matrix may be desired when potencies between desired biomodulatory molecules are substantially different from one another. In this example two matrices each comprising different biomodulatory molecules are constructed such that each may be administered in different amounts or in different locations. Preferably a combination matrix is formed from biomodulatory molecules that provide a beneficial accumulatory effect when administered simultaneously. For example, proinflammatory molecules such as TNF, IL-1, IL-6, CpG sequences, SEB, endotoxin, or other bacterial molecules may be useful in combination to provide a stimulatory effect, and antiinflammatory molecules such as IL-10 and TGF-β may be useful in a combination to provide immune suppression (The Cytokine Handbook 3^{rd} Ed., 1998 Ed. Angus Thompson Academic Press). The preferred form of the invention is to use a single type of biomodulatory molecule such as GM-CSF at a molar amount sufficient for activation of the immune system.

Alternatively, a bioactive molecular matrix may be attached to a polymeric support or to each other and to a polymeric support. Supports that may be utilized by the present invention include for example agarose, gelatins, microspheres and gels. These supports are preferably biodegradable, bioerodible or resorbable. If a porous polymeric support is being utilized the biomodulatory molecule is anchored to the polymeric support then antigen is captured and retained within the support. The diameter of the pores required for this function will vary with the molecular weight of the antigen. In another embodiment of the invention, the antigen can also be anchored to the polymeric support through biochemical conjugation chemistry. Polymeric agents that may be utilized with the present invention include Gelfoam™ (Pharmacia, Kalamazoo, Mich.), gelatin and alginate, agarose. In the gel configuration, the display of the modulatory molecule is generally stationary and the antigen is encapsulated or anchored within the gel.

The chemistries used for proteinaceous biomodulatory conjugation to the polymeric support involve coupling an amine group from amino acid residues of the biomodulatory protein to the activated polymeric support. The polymeric support may be activated by the addition of any group able to be coupled to an amine group such as a carboxyl or a hydrazide. Preferably the polymeric support is activated by the addition of an aldehyde. Alternatively, the polymeric support may be activated by the addition of an amine group and the biomodulatory protein comprising the complimentary binding group. Furthermore, minor variations in conjugation conditions may be necessary depending on the individual molecules.

The conjugation chemistry for CpG sequences comprise chemically modifying the oligonucleotide to incorporate any coupling group such as a diamine group. Any group able to be conjugated to a CpG sequence and able to be coupled to an aldehyde would be useful in the present invention.

When two or more different types of proteinaceous biomodulatory molecules are used in conjunction with one another or when two or more different types of nucleic acid sequences are used in conjunction with one another, the order of biomodulatory molecule conjugation is not imperative. The preferred method involves simultaneous conjugation of generally equimolar amounts of each desired biomodulatory molecule. However the molar amounts may vary depending on the affinity the molecule has to its target. Determination of the molar amount of each may be performed by a modified ELISA-based immunoassay specific for each of the molecules. The optimum molar amount of each is then evaluated based on the optimal immune response generated against a desired antigen. These amounts are then used in the formation of the biomodulatory matrix.

When a proteinaceous biomodulatory molecule is used in conjunction with a nucleic acid biomodulatory molecule, the conjugation chemistries may require each to be conjugated separately. In this configuration the conjugation of the nucleic acid sequence is performed prior to conjugation of the proteinaceous sequence. However different conjugation chemistries may allow the simulataneous conjugation of the nucleic acid and protein molecules.

A preferred biomodulatory matrix will retain bioactivity of the biomodulatory molecules comprising the matrix, will be able to capture, encapsulate, associate and/or conjugate with a wide variety of other molecules such as disease-associated antigens, will not initiate a detrimental immune response against itself and will not cause a serious inflammatory response when administered to a host.

The retention of bioactivity may be tested by performing standard activity assays unique for each biomodulatory molecule comprising the matrix. For example, cytokine molecules attached to a matrix may be assayed for their ability to induce the proliferation of bone marrow cells or activation of other cells in accordance with their known function. GM-CSF attached to a matrix may be assayed by incubation with bone marrow cells as detailed in Cytokines, A Practical Approach, 2nd Edition, F. R. Balkwill, Ed. 1995 IRL Press. For bacterial LPS, the limulus amebocyte lysate assay (LAL) is a sensitive assay used to detect quantitative amounts of endotoxins or bacterial lipid A. Cellular immunity assays to purified antigen such as T cell proliferation and cytoxicity assays may also be used to test the retention of bioactivity.

Molecular capture and encapsulation by or association and conjugation of the matrix may be determined by a variety of methods. Once the matrix has been formed its composition may be determined using methods that identify molecules or fragments of molecules contained in a composition by immunoassay, chromatographic, electrophoretic or mass spectrographic techniques following degradation, digestion and/or electronic bombardment. In particular, bone marrow proliferation or cytokine assays are used to measure activity of the matrix against immune accessory cells, and preferably ELISA and BCA (Pierce Chemical Co., Rockford, Ill.) are used to determine the composition of the matrix. Moreover, the absence of soluble biomodulatory molecules may be demonstrated by ELISA analysis of the supernatant from a pelleted bioactive matrix. ELISA and BCA analysis of the supernatant from a pelleted bioactive matrix may also be used to determine the quantity of molecule encapsulated by subtracting the supernatant molar amount from the known added molar amount.

Immune and inflammatory responses may be one of the adverse responses in patients caused by this immunomodulatory matrix. Since one of the main routes of administration will be subcutaneous or intradermal injection, one trained in the art could observe induration and erythema responses by measuring specific and nonspecific antibody concentrations from serum using ELISA, and measuring cellular proliferation using flow cytometry techniques.

In one embodiment, the invention is a multicomponent system wherein the matrix further comprises a disease-associated antigen or immunogenic epitope of a disease-associated antigen covalently bound to the matrix or coadministered as a combined bolus or cocktail. In fact any disease-associated antigen that would be effective in mounting an immune response against that antigen in a host would be beneficial in the present invention. Such antigens can be endogenous or exogenous to the cell and include, for example, tumor-associated antigens, autoimmune disease-associated antigens, disease-associated antigens, viral antigens, bacterial antigens parasitic antigens, whole tumor cells inactivated by irradiation or other comparable method, and lysates of tumor cells.

A variety of tumor-associated antigens may be utilized in the present invention and include those that are tumor specific as well as those that are tumor selective. Tumor associated antigens include p53 and mutants thereof, Ras and mutants thereof, HER-2/Neu, EGFR, EGFRvIII, bcr/abl breakpoint peptides, carcinoembryonic antigen, MUC-1, minor histocompatability HLA-A2, MAGE-1, MAGE-3, BAGE, GAGE-1, GAGE-2, MART-1/MelanA, gp100, tyrosinase, TRP-1, MUM-1, β-catenin, CDK-4, p15, N-acetylglucosaminyltransferase-V, HPV E6 and HPV E7. Purified antigen as well as tumor cell lysates would also be useful with the present invention.

A tumor-associated antigen may be an oncogenic protein such as a non-mutated, overexpressed oncoprotein or a mutated, unique oncoprotein (Disis and Cheever, Current Opin. Immunol. 8:637 (1996); Cornelis et al., Curr. Opin. Immunol. 8:651 (1996)). For example, mutations in p53 are present in about 50% of human malignancies. This mutant p53 protein or a peptide fragment thereof may be utilized in the present invention (Yanuck et al., Cancer Res. 53:3257 (1993); Noguchi et al., PNAS 92:2219 (1995)). In addition, the wild type p53 protein or a peptide fragment thereof may also be utilized in the present invention. (Theobald et al., PNAS 92:11993 (1995); Houbiers et al., Immunol. 23:2072 (1993)). Although p53 is present in both normal and tumor cells, matrices including wild type p53 peptide can promote a selective immune response against tumor cells due to the relative increased accumulation of p53 in the cytosol of tumor cells.

Mutations in Ras are present in about 15% of human malignancies. Mutant Ras protein and peptide fragments thereof can be tumor-associated antigens useful in the present invention for treating such malignancies. Mutant Ras proteins usually have a single amino acid substitution at residue 12 or 61. Ras peptides spanning this mutant segment can be useful tumor-associated antigens (Cheeveer et al., Immunol. Rev. 145:33 (1995): Gjertsen et al., Lancet 346:1399 (1995); Abrams et al., Seminars Oncol. 23:118 (1996); Abrams et al., Eur. J. Immunol. 26:435 (1996)).

HER-2/neu is a growth factor receptor overexpressed in about 30% of breast and ovarian cancers as well as in a wide variety of other adenocarcinomas. HER-2/neu and peptides derived from the HER-2/neu proto-oncogene are tumor-associated antigens that can be useful in the present invention (Disis et al., Cancer Res. 54:1071 (1994); Bernhard et al., Cancer Res. 55:1099 (19950; Mayordomo et al., Nature 1:1297 (1995) Pegram M, Slamon D, Seminars Oncol. 27(5 Suppl 9):13 (2000); Angus et al., Seminars Oncol. 27 (6 Suppl 11): 53 (2000)).

Epidermal growth factor receptor (EGFR) or an immunogenic epitope thereof or a mutant EGFR variant or immunogenic epitope thereof are tumor-associated antigens useful in the present invention. For example, the EGFR deletion mutant EGFRvIII is expressed in a subset of breast carcinomas and in non-small cell lung carcinomas and malignant gliomas. EGFRvIII disease-associated antigens, such as peptides corresponding to the novel EGFRvIII fusion junction, can be useful in stimulating an immune response against such tumors (Wikstrand et al., Cancer Res. 55:3140 (1995); Moscatello et al., Cancer Res. 57:1419 (1997)). Consequently, EGFR and EGFRvIII disease-associated antigens or immunogenic epitopes thereof can be useful in the present invention for the treatment of malignant gliomas, breast and lung carcinomas and to protect individuals at high risk from developing these cancers.

A tumor-associated antigen can also be a joining region segment of a chimeric oncoprotein such as bcr/abl (Ten-Bosch et al., Leukemia 9:1344 (1995); Ten-Bosch et al., Blood 87:3587 (1996)). This chimeric oncoprotein is present in chronic myeloid leukemia. Consequently, an antigen corresponding to this joining region segment can be useful in the present invention for the treatment of CML.

Carcinoembryonic antigen (CEA) is highly expressed in the majority of colorectal, gastric and pancreatic carcinomas (Tsang et al., J. Natl. Cancer Inst. 87:982 (1995)) and may also be a useful tumor-associated antigen in the present invention.

The MUC-1 mucin gene product which is an integral membrane glycoprotein present on epithelial cells, also is a tumor-associated antigen useful in the present invention. Mucin is express in almost all human epithelial cell adenocarcinomas, including breast, ovarian, pancreatic, lung, urinary bladder, prostate and endometrial carcinomas, representing more than half of all human tumors. (see Fin et al., Immunol. Rev. 145:61 (1995); Barratt-Boyes, Cancer Immunol. Immunother. 43:142 (1996)). Matrixes of the present invention containing full length mucin or immunogenic epitopes thereof can therefore be used to protect against or treat epithelial cell adenocarcinomas such as breast carcinomas (Lalani et al., J. Biol. Chem. 266:15420 (1991)).

Minor histocompatibility antigens may also be utilized in the present invention (Goulmy Curr. Opin. Immunol. 8:75 (1996): Den Haan et al., Science 268:1478 (1995); Wang et al., Science 269:1588 (1995)). Major histocompatibility complex antigens, when mutated in some tumors, may also be utilized in the present invention. For example, an HLA-A2 antigen having a single amino acid substitution can be used in the present invention to treat human renal cell carcinomas (Brandle et al., J. Exp. Med. 183:2501 (1996)).

A variety of widely shared melanoma antigens may also be tumor-associated antigens useful with the present invention (Robbins and Kawakami Curr. Opin. Immunol. 8:628 (1996): Celli and Cole, Seminars Oncol. 23:754 (1996)). For example, the MAGE-1, MAGE-2, MAGE-3, BAGE, GAGE-1 and GAGE-2 tumor-associated antigens or immunogenic epitopes thereof such as MZ2-E can be utilized with the present invention for protection against melanoma (van der Bruggen Science 254; 1643 (1991)). In normal adult tissue, the expression of MAGE related gene products are limited to testes and placenta; however, these tumor-associated antigens are expressed in a wide variety of tumor types, including breast carcinomas and sarcomas. A widely expressed melanoma tumor-associated antigen useful with the present invention is N-acetylglucosaminyltransferase-V which is expressed at significant levels in about 50% of melanomas and absent in normal tissues (Guilloux et al., J. Exp. Med. 183:1173 (1996)).

Melanoma tumor-associated antigens may also be differentiation antigens expressed by normal melanocytes. Such melanoma tumor-associated antigens include MART-1/MelanA, gp100, tyrosinase, the key enzyme in pigment synthesis and the tyrosinase-related protein TRP-1 (gp75).

Unique melanoma antigens such as MUM-1, β-catenin and cyclin-dependent kinase CDK4 melanoma antigens (Coulie et al., PNAS 92:7976 (1995); Wolfel et al., Science 269:1281 (1995); Robbins et al. J. Exp. Med. 183:1185 (1996)) may be tumor-associated antigens useful in the present invention (Mumberg et al., Seminars in Immunol. 8:289 (1996)).

The matrixes of the invention may also contain autoimmune disease-associated antigens and can be useful in protecting against or treating diseases such as rheumatoid arthritis, psoriasis, multiple sclerosis, systemic lupus erythromatosus and Hashimoto's disease, type I diabetes mellitus, myasthenia gravis, Addison's disease, autoimmune gastritis, Grave's disease and vitiligo. Autoimmune disease-associated antigens useful in the invention include, for example, T cell receptor derived peptides, such as Vβ14, Vβ3, Vβ17, Vβ13 and Vβ6 derived peptides and annexins such as AX-1, AX-2, AX-3, AX-4, AX-5 and AX-6 which are autoantigens associated with autoimmune diseases such as systemic lupus erythematosus, rheumatoid arthritis and inflammatory bowel disease (Bastain Cell. Mol. Life. Sci. 53:554 (1997)). In addition, the annexins may be tumor-associated antigens useful in the present invention.

A variety of other disease-associated antigens can also be included in the present invention. Such disease-associated antigens include viral, parasitic, yeast and bacterial antigens. For example, Helicobacter pylori (H. pylori) is the major causative agent of superficial gastritis and plays a central role in the etiology of peptic ulcer disease. Infection with H. pylori also appears to increase the risk of gastric cancer. The present invention can be useful in protecting against H. pylori infection. Such matrixes can contain an H. pylori disease-associated antigen, for example, the urease protein, 90 kDa vacuolating cytotoxin (VacA), or 120 to 140 kDa immunodominant protein (CagA) of H. pylori, or immunogenic epitopes thereof (Clyne and Drumm Infect. Immun. 64:2817 (1996); Ricci et al., Infect. Immun. 64:2829 (1996)).

A viral disease-associated antigen useful in the invention can be a human immunodeficiency virus type I (HIV-1) antigen. Such antigens include the gp120 envelope glycoprotein and immunogenic epitopes thereof such as the principal neutralization immunogenic determinant (PND), gp160 and HIV-1 core protein derived immunogenic epitopes (Ellis (Eds.) Vaccines: New Approaches to Immunological Problems Stoneham, Mass.; Reed Publishing Inc. (1992)). Another viral disease-associated antigen useful in the invention can be any of the influenza viruses including, but not limited to, H1N1, H5N1, as well as avian influenza subtypes and serotypes known in the art. Both viral protein subunits as well as whole, inactivated viruses would be useful as antigens for this invention. Furthermore, proteins derived from hepatitis C or B viruses (HCV, HBV) would be useful antigens in this invention. Both recombinantly produced in yeast or other organism and whole inactivated viruses can serve as appropriate antigens in this invention. For example, the S antigen of HBV or core antigens of hepatitis virus would be useful antigens.

Additional disease-associated antigens useful in the present invention include the MP65 antigen of Candida albicans (Gomez et al., Infect. Immun. 64:2577 (1996)); helminth antigens; Mycobacterial antigens including M. bovis and M. tuberculosis antigens; Haemophilus antigens; Pertussis antigens; respiratory syncytial viral antigens, poliovirus antigens, herpes simplex virus antigens; rotavirus antigens and flavivirus antigens (Ellis supra (1992)).

Exemplary Disease-associated Antigens

| | | |
|---|---|---|
| Antigen | Epitope | Reference |

Non-melanoma antigens

| | | |
|---|---|---|
| HER-2/neu | IISAVVGIL | (SEQ ID NO. 2) |
| | | Peoples et al., PNAS |
| | | 92:432 (1995) |
| | KIFGSLAFL | (SEQ ID NO. 3) |
| | | Fisk et al., J. Exp. Med. |
| | | 181:2109 (1995) |
| HPV E6, E7 | YMLDLQPETT | (SEQ ID NO. 4) |
| | | Ressing et al., Cancer |
| | | Res. |
| | | 56:582 (1996) |
| MUC-1 | PDTRPAPGSTAPP | (SEQ ID NO. 5) |
| | AHGVTSA | Fin et al., Immunol. Rev. |
| | | 145:61 (1995) |

Tumor-specific, widely shared antigens

| | | |
|---|---|---|
| MAGE-1 | EADPTGHSY | (SEQ ID NO. 6) |
| | | Traversari et al., J. Exp. |
| | | Med. |
| | | 176:1453 (1992) |
| | SAYGEPRKL | (SEQ ID NO. 7) |
| | | van der Bruggen et al., |
| | | Eur. J. Immunol. |
| | | 24:2 134 (1994) |
| MAGE-3 | EVDPIGHLY | (SEQ ID NO. 8) |
| | | Gaugler et al., J. Exp. |
| | | Med. |
| | | 179:921 (1994) |
| | FLWGPRALV | (SEQ ID NO. 9) |
| | | Celis et al., PNAS |
| | | 91:2105 (1994) |
| BAGE | AARAVFLAL | (SEQ ID NO. 10) |
| | | Boel et al., Immunity |
| | | 2:167 (1995) |
| GAGE-1, 2 | YRPRPRRY | (SEQ ID NO. 11) |
| | | Van den Eynde et al., J. |
| | | Exp. Med. |
| | | 182:689 (1995) |
| GnT-V | VLPDVFIRC | (SEQ ID NO. 12) |
| | | Guilloux et al., J. Exp. |
| | | Med. |
| | | 183:1173 (1996) |
| p15 | AYGLDFYIL | (SEQ ID NO. 13) |
| | | Robbins et al., J. |
| | | Immunol. |
| | | 154:5944 (1995) |

Melanocyte lineage proteins

| | | |
|---|---|---|
| gp100 | KTWGQYWQV | (SEQ ID NO. 14) |
| | ITDQVPFSV | (SEQ ID NO. 15) |
| | YLEPGPVTA | (SEQ ID NO. 16) |
| | LLDGTATLRL | (SEQ ID NO. 17) |
| | VLYRYGSFSV | (SEQ ID NO. 18) |
| | | Kawakami et al., J. |
| | | Immunol. |
| | | 154:3961 (1995) |
| MART-1/ | AAGIGILTV | (SEQ ID NO. 19) |
| MelanA | | Kawakami et al., J. Exp. |
| | | Med. |
| | | 180:347 (1994) |
| | ILTVILGVL | (SEQ ID NO. 20) |
| | | Castelli et al., J. Exp. |
| | | Med. |
| | | 181:363 (1995) |
| TRP-1 | MSLQRQFLR | (SEQ ID NO. 21) |
| (gp75) | | Wang et al., J. Exp. Med. |
| | | 183:1131 (1996) |
| Tyrosinase | MLLAVLYCL | (SEQ ID NO. 22) |
| | | Wolfel et al., Eur. J. |
| | | Immunol. |
| | | 24:759 (1994) |
| | YMNGTMSQV | (SEQ ID NO. 23) |
| | | Wolfel et al., supra |
| | | (1994) |
| | SEIWRDIDF | (SEQ ID NO. 24) |
| | | Brichard et al., J. |
| | | Immunol. |
| | | 26:224 (1996) |
| | AFLPWHRLF | (SEQ ID NO. 25) |
| | | Kang et al., J. Immunol. |
| | | 155:1343 (1995) |
| | QNILLSNAPLGPQ | (SEQ ID NO. 26) |
| | SYLQDSDPDSFQD | (SEQ ID NO. 27) |
| | | Topalian et al., J. Exp. |
| | | Med. |
| | | 183:1965 (1996) |

Tumor-specific antigens

| | | |
|---|---|---|
| β-catenin | SYLDSGIHF | (SEQ ID NO. 28) |
| | | Robbins et al., J. Exp. |
| | | Med. |
| | | 183:1185 (1996) |
| MUM-1 | EEKLIVVLF | (SEQ ID NO. 29) |
| | | Coulie et al., PNAS |
| | | 92:7976 (1995) |
| CDK4 | ACDPHSGHFV | (SEQ ID NO. 30) |
| | | Wolfel et al., Science |
| | | 269:1281 (1995) |

The methods of the invention for modulating an immune response can be used to treat a variety of diseases, conditions and disorders including tumors and cancers, autoimmune diseases, infectious diseases, and disorders of bacterial, parasitic and viral etiology. In one embodiment, the methods of the invention can be used to modulate an immune response for protection against or treatment of cancer including cancers such as melanoma, colorectal cancer, prostate cancer, breast cancer, ovarian cancer, cervical cancer, endometrial cancer, glioblastoma, renal cancer, bladder cancer, gastric cancer, pancreatic cancer, neuroblastoma, lung cancer, leukemia and lymphoma. The methods of the invention may also be used to protect against or treat infectious diseases such as influenza, SARS, hepatitis B, hepatitis C, Acquired Immunodeficiency Syndrome (AIDS), tularemia, anthrax, West Nile virus, listeria, tuberculosis,

In addition, the methods of the invention can be used to protect against the development of or to treat existing autoimmune diseases such as rheumatoid arthritis, psoriasis, multiple sclerosis, systemic lupus erythromatosus and Hashimoto's disease, type I diabetes mellitus, myasthenia gravis, Addison's disease, autoimmune gastritis, Grave's disease and vitiligo. Allergic reactions, such as hay fever, asthma, systemic anaphylaxis or contact dermatitis may also be treated using the methods of the invention for modulating an immune response.

A variety of diseases or conditions of bacterial, parasitic, yeast or viral etiology may also be interfered with, prevented and/or treated using the methods and compositions of the present invention for modulating an immune response. Such diseases include gastritis and peptic ulcer disease, periodontal disease, Candida infections, helminthic infections, tuberculosis, Hemophilus-mediated disease such as whooping cough, cholera, malaria, influenza infections, respiratory syncytial disease, hepatitis, poliomyelitis, genital and non-genital herpes simplex virus infections, rotavirus-mediated conditions, such as acute infantile gastroenteritis and diarrhea and flavivirus-mediated diseases such as yellow fever, encephalitis, papiloma viruses, syncia viruses, varicella viruses, cytomegalovirus, Epstein Barr Virus, Herpes Simplex Virus (HSV), Coxsakie Virus, Corona Virus. As disclosed herein the methods of the invention can be used to therapeutically treat an individual having, or suspected of having, one of such diseases or conditions. In addition, the methods of the present invention may be used to protect an individual who is at risk for developing one of such diseases or conditions from the onset of the actual disease. Individuals predisposed to developing, for example, melanoma, retinoblastoma, breast cancer or colon cancer or disposed to developing multiple sclerosis or rheumatoid arthritis can be identified using methods of genetic screening (Mao et al., Canc. Res. 54 (suppl):1939s-1940s (1994); Garber and Diller, Curr. Opin. Pediatr. 5:712 (1993)).

The matrix of the invention may be administered with a variety of pharmaceutically acceptable carriers such as for example, water and isotonic saline solutions which are preferably buffered at physiological pH (such as phosphate-buffered saline or TRIS-buffered saline). Preferably the pharmaceutical carrier is physiological saline.

Administration can be accomplished by any number of a variety of methods including subcutaneous, intradermal or intramuscular injection and injection directly into tumor lesions. For treatment of tumors administration can be at a location other than the primary tumor site. Multiple routes of administration, as well as administration at multiple sites to increase the area contacted by the matrix are also envisioned by the present invention. Moreover matrices comprising different modulatory molecules may be administered using different methods or in different locations. It is recognized that boosters administered, for example, every several months, may also be useful in modulating an immune response against a disease-associated antigen according to the methods of the invention.

The effectiveness of therapy can be determined by monitoring immune function in a patient. In anti-tumor therapy, for example, the cytolytic activity of immune effector cells against a patient's cancer cells can be assayed using methods known in the art. In addition, the size or growth rate of a tumor can be monitored in vivo using methods of diagnostic imaging. By monitoring the patient during therapy, the physician will be able to assess whether to use repeated administration of the matrix of the invention. For immunity against infectious diseases, effectiveness can be measured by determining the immunogenicity of antigens when incubated with peripheral blood cells of the patient in a standard proliferation assay. Furthermore, evaluation of antigen-specific antibodies in the immunoglobulin fraction of patient sera can determine whether a strong humoral immune response has been generated against the antigens.

The following examples are offered by way of illustration and not by way of limitation.

### EXAMPLES

### Example 1

### Procedure for Activation of Dextran into Polyaldehyde Dextran

Approximately 6.42 grams of sodium periodate (NaIO₄, Sigma, St. Louis, Mo.) is dissolved in 500 mL of deionized water to a concentration of 30 mM. Dissolve dextran (Molecular weight 10,000-40,000 Polysciences, Warrington, Pa.) in the sodium periodate solution with constant stirring and allow to react overnight in the dark at room temperature. Remove sodium periodate by dialysis against water. The polyaldehyde dextran may be lyophilized and stored at 0-4° C. The efficiency of the oxidation of dextran may be determined by reduction of Cu²⁺ to Cu⁺ (described by Smith P. et al. Anal. Biochem. 150:76, 1985) The amount of Cu⁺ formed is proportional to the amount of aldehyde groups present on the surface of the dextran.

### Example 2

### Procedure for the Coupling of GM-CSF to Activated (Polyaldehyde) Dextran

Dissolve the activated (polyaldehyde) dextran or buffer-exchange the activated dextran in periodate solution into 100 mM sodium phosphate, 150 mM NaCl, pH 7.2 with constant stirring to a concentration of about 10-15 mg/mL. To this mixture add 1 mg of GM-CSF (ratio of 1:1 v/v) that had been previously dialyzed into the reaction buffer (i.e. 100 mM sodium phosphate, 150 mM NaCl, pH 7.2). To this solution is added 200 μl of 1 M cyanoborohydride (Aldrich, Milwaukee, Wis.) and the mixture is allowed to react for six hours at room temperature. The remaining unreacted aldehyde groups on the dextran are blocked by adding 200 μl of 1 M Tris buffer, pH 8 and incubating the mixture an additional 2 hours at room temperature. The GM-CSF conjugated dextran is purified by passing this mixture through a Sephacryl S-200 or S-300.

### Example 3

### Characterization of the GM-CSF/Dextran Conjugate

A. Characterization of Bound GM-CSF by Inhibition ELISA

A 96 well polystyrene microtiter plate (Falcon, Becton Dickinson Labware, Lincoln Park, N.J.) is coated with 50 μl of purified anti-mouse GM-CSF (10 μg/mL in phosphate buffered saline (PBS)) and allowed to react over night at 4° C. Excess solution is removed, and the wells are blocked with blocking solution (100 uL of 1% FBS in PBS) for 1 hour at room temperature. Excess blocking solution is removed. 50 μL of nanoparticles (with or without GM-CSF) are added to the wells and incubated for 30 minutes at room temperature. Wells are washed three times with blocking buffer. A second anti-mouse GM-CSF antibody (recognizing a separate epitope and labeled with horse-radish peroxidase) is added (10 μg/mL in blocking solution) and incubated for 30 minutes at room temperature. Wells are again washed three times with blocking buffer. 50 μL substrate (Tetramethylbenzidine, Kirkegard and Perry Bethesda, Md.) is added to the wells and developed for 5 minutes, stopped with 50 μL 1M H₃PO₄ and read on an ELISA plate reader at 550-650 nm.

B. Protein Concentration Determination by BCA

The BCA (Pierce, Rockford, Ill.) assay should be followed according to the instructions in the kit. Briefly protein concentration is determined by detecting the reduction of Cu²⁺ to Cu¹⁺ by bicinchoninic acid in the presence of protein comprising cysteine, cystine, tryptophan, and tyrosine residues and comparing the absorbance at 562 nm using a spectrophotometer and comparing the results to a standard curve constructed from bovine serum albumin (BSA).

C. Characterization of Biomodulatory Matrix Activity by Bone Marrow Proliferation Assay

Various set amounts of GM-CSF/dextran conjugate are incubated with 10³ isolated mouse bone marrow cells (DBA/2, C57BL/6, or BALB/c) in a 96 well microtiter plate with a final volume in media of 200 μl. Two days after cultures are initiated microtiter wells are pulsed with 30 μCi of ³H-thymidine (ICN, Costa Mesa, Calif.) and incubated overnight in a CO₂ humidified chamber at 37° C. Microtiter wells are harvested and counts per minute (cpm) determined.

Alternatively, proliferation can be measured without the use of radioisotope reagents in the following manner. The bone marrow cells are washed and resuspended in complete media (RPMI 1640, 10% FBS, GlutaMAX™, 5 μM β-ME), and incubated at final volume of 200 μl (3-5×10⁵ cells per well) in flat-bottom, opaque white-wall plates for 72-96 hours at 37° C. and 5% CO₂. Four to Sixteen hours prior to harvest, the cells would be pulsed with 10 μM BrdU and processed according to the procedures for the Delfia Proliferation Assay (Perkin-Elmer, Wellesley, Mass.). Anti-BrdU Europium-based fluorescence is detected using a Wallac-1420 Victor-2 time-resolved fluorimeter. Results are represented as relative fluorescence units (RFU)±standard error of the mean (SEM).

### Example 4

### Administration of the GM-CSF Conjugate to Mice

Increasing known amounts of GM-CSF/Dextran conjugate are admixed with 10⁶ irradiated mouse mastocytoma cells (P815) and injected intradermally into the hind flanks of 6 to 8 week old female mice (DBA/2). The animals are injected twice (i.e. primed and boosted) over a period of two weeks and then challenged with 106 unirradiated P815 cells in the opposite flank subcutaneously. Animals are monitored daily and tumor size measured with a micrometer. Tumor growth is evaluated in animals compared with control animals who received no treatment.

### Example 5

### Procedure for the Coupling of GM-CSF and IL-2 to Activated (Polyaldehyde) Dextran

Dissolve the activated (polyaldehyde) dextran or buffer-exchange the activated dextran in periodate solution into 100 mM sodium phosphate, 150 mM NaCl, pH 7.2 with constant stirring to a concentration of about 10-15 mg/mL. To this mixture add 1 mg of GM-CSF (ratio of 1:1 v/v) and 1 mg of IL-2 that had each been previously dialyzed into the reaction buffer (i.e. 100 mM sodium phosphate, 150 mM NaCl, pH 7.2). To this solution is added 200 μl of 1 M cyanoborohydride (Aldrich, Milwaukee, Wis.) and the mixture is allowed to react for six hours at room temperature. The remaining unreacted aldehyde groups on the dextran are blocked by adding 200 μl of 1 M Tris buffer, pH 8 and incubating the mixture an additional 2 hours at room temperature. The GM-CSF and IL-2 conjugated dextran is purified by passing this mixture through a Sephacryl S-200 or S-300.

### Example 6

### Characterization of the GM-CSF/IL-2/Dextran Conjugate

A. Characterization of Bound GM-CSF by Inhibition ELISA is performed as in Example 3 above.

B. Characterization of Bound IL-2

Bound IL-2 will be characterized using a tandem antibody (anti-IL2) assay commercially available (R&D Systems, Minneapolis, Minn.). Briefly, conjugated IL-2 matrices are added to the wells of a 96-well ELISA plate containing primary anti-IL-2 antibodies. This is allowed to incubate for 1 hour at room temperature. The wells are then washed with wash buffer and the secondary anti-IL-2 antibody added and incubated for 1 hour at room temperature. Finally, the amount of IL-2 captured in the wells is detected using a chromogen labeled (horse radish peroxidase or equivalent) antibody to bind to the secondary antibody. The resulting reaction is read on a ELISA plate reader.

Alternatively, the supernatants containing unconjugated IL-2 can be analyzed using the same assay in order to quantitate the amount of IL-2 unbound in a method to indirectly measure the efficiency of conjugation.

C. Protein Concentration Determination by BCA

The BCA (Pierce, Rockford, Ill.) assay should be followed according to the instructions in the kit. Briefly protein concentration is determined by detecting the reduction of Cu²⁺ to Cu¹⁺ by bicinchoninic acid in the presence of protein comprising cysteine, cystine, tryptophan, and tyrosine residues and comparing the absorbance at 562 nm using a spectrophotometer and comparing the results to a standard curve constructed from BSA.

D. Characterization of Biomodulatory Matrix Activity by Bone Marrow Proliferation Assay

Various set amounts of GM-CSF/IL-2/dextran conjugate are incubated with 10³ isolated mouse bone marrow cells (DBA/2) in a 96 well microtiter plate with a final volume in media of 200 μl. Two days after cultures are initiated microtiter wells are pulsed with 30 μCi of ³H-thymidine (ICN, Costa Mesa, Calif.) and incubated overnight in a CO₂ humidified chamber at 37° C. Microtiter wells are harvested and CPM determined. An alternative non-isotopic assay can also be used and is described above in Example 3.C.

### Example 7

### Procedure for Activation of Ficoll into Polyaldehyde Ficoll

Approximately 6.4 grams of Sodium periodate (Sigma, St. Louis, Mo.) is dissolved in 500 mL deionized water to a concentration of about 30 mM. Ficoll (Molecular weight 10,000-40,000) is dissolved in the sodium periodate solution with constant stirring and allowed to react overnight in the dark at room temperature. The periodate is then removed by dialysis against water and the dialyzed polyaldehyde Ficoll may lyophilized and stored. The efficiency of the oxidation of dextran may be determined by reduction of Cu²⁺ to Cu¹⁺ (described by Smith P. et al. Anal. Biochem. 150:76, 1985) The amount of Cu⁺ formed is proportional to the amount of aldehyde groups present on the surface of the Ficoll.

### Example 8

### Procedure for the Coupling of GM-CSF to Ficoll

The lyophilized polyaldehyde/Ficoll is dissolved or the periodate-oxidized Ficoll in periodate solution is buffer-exchanged into 100 mM sodium phosphate, 150 mM NaCl, pH 7.2 with constant stirring to a concentration of about 10-25 mg/mL. To this mixture is added 1 mg of GM-CSF (ratio of 1:1 v/v) that had been previously dialyzed into the reaction buffer (i.e. 100 mM sodium phosphate, 150 mM NaCl, pH 7.2). To this solution is added 200 μl of 1 M cyanoborohydride (Aldrich, Milwaukee, Wis.) and the mixture is allowed to react for six hours at room temperature. The remaining unreacted aldehyde groups on the dextran are blocked by adding 200 ml of 1 M Tris buffer, pH 8 and incubating the mixture an additional 2 hours at room temperature. The GM-CSF conjugated dextran is purified by passing this mixture through a Sephacryl S-200 or S-300.

### Example 9

### Characterization of the GM-CSF/Ficoll Conjugate

A. Characterization of Bound GM-CSF by Inhibition ELISA

A 96 well microtiter plate is coated with 50 μl of purified mouse GM-CSF (10 μg/mL in phosphate buffered saline “PBS”) and allowed to react over night at 4° C. Excess solution is removed, and the wells are blocked with blocking solution (100 μL of 1% FBS in PBS) for 1 hour at room temperature. Excess blocking solution is removed. 50 μL of ficoll conjugate (with or without GM-CSF) is added to the wells and incubated for 30 minutes at room temperature. Wells are washed three times with blocking buffer. A second anti-mouse GM-CSF antibody (recognizing a separate epitope and labeled with horse-radish peroxidase) is added (10 μg/mL in blocking solution) and incubated for 30 minutes at room temperature. Wells are again washed three times with blocking buffer. 50 μL substrate (Tetramethylbenzidine, Kirkegard and Perry Bethesda, Md.) is added to the wells and developed for 5 minutes, stopped with 50 μL 1 M H₃PO₄ and read on an ELISA plate reader at 550-650 nm.

B. Protein Concentration Determination by BCA

The BCA (Pierce, Rockford, Ill.) assay should be followed according to the instructions in the kit. Briefly protein concentration is determined by detecting the reduction of Cu²⁺ to Cu¹⁺ by bicinchoninic acid in the presence of protein comprising cysteine, cystine, tryptophan, or tyrosine residues and comparing the absorbance at 562 nm using a spectrophotometer and comparing the results to a standard curve constructed from BSA.

C. Characterization of Biomodulatory Matrix Activity by Bone Marrow Proliferation Assay

Various set amounts of GM-CSF/Ficoll conjugate are incubated with 10³ isolated mouse bone marrow cells (DBA/2) in a 96 well microtiter plate with a final volume in media of 200 μl. Two days after cultures are initiated microtiter wells are pulsed with 30 μCi of ³H-thymidine (ICN, Costa Mesa, Calif.) and incubated overnight in a CO₂ humidified chamber at 37° C. Microtiter wells are harvested and CPM determined. An alternative non-isotopic assay can also be used and is described above in Example 3.C.

### Example 10

### Procedure for Formation of Microspheres Conjugated with Bioactive Molecules

Polymers of poly-(D,L-lactide-co-glycolide), (“PLG”) and poly-(L-lactic acid), (“PLA”) are conjugated with GM-CSF or CpG oligonucleotides using chemistries similar to those mentioned previously and prepared as microspheres. Briefly, solvent evaporation microspheres were prepared by homogenizing 1.0 ml phosphate buffered saline (“PBS”) with 5.0 ml of a 6% (w/v) solution of PLG or PLA in dichloromethane for 1 minute using an ultraturrax (TP-18-10, Ika-Werk, Staufen, Germany). The resulting mixture was then decanted into a solution of 10% polyvinyl alcohol in PBS and homogenized 1 minute. The mixture was then magnetically stirred overnight to allow for evaporation and sphere formation. The microspheres were then collected by centrifugation at 17,500×g for 10 minutes, washed twice with PBS and resuspended in 5.0 mls PBS. This procedure is also performed in the presence of antigen for the encapsulation of antigen into the forming spheres.

### Example 11

### Modification of CpG Oligonulceotides or Diphosphoryl Lipid A (DPLA) for Coupling to PLG-PLA Copolymers

Oligonucleotides (5′-TCC ATG ACG TTC CTG ATG CT-3′) (SEQ ID NO. 1) or DPLA is modified with carbodiimide EDC in the presence of imidazole for 30 minutes at room temperature. Resulting product is purified by gel filtration on Sephadex G-25 using 10 mM sodium phosphate, 0.15 M NaCL, 10 mM EDTA, pH 7.2 and dialyzed into DMSO.

### Example 12

### Procedure for Coupling CpG Oligonucleotide or Diphosphoryl Lipid A (DPLA) to PLG-PLA Copolymers

Activated PLG-PLA (by adding 1 g PLG-PLA, 9 mg carbodiimide DCC in 3 mL of dimethyl sulfoxide (DMSO) at room temperature for 2 hours) is slowly dropped into 5 mL of DMSO containing the modified oligonucleotides or modified DPLA and incubated for 3 hours. The resulting conjugate is slowly dropped into an excess of diethyl ether, washed with dionized water and then lyophilized.

### Example 13

### Procedure for Matrix Formation of CpG—PLG-PLA or DPLA—PLG-PLA Presenting CpG Sequences or DPLA and the Encapsulation of Tumor Associated Antigen

Polymer conjugates (either CpG-PLG-PLA or DPLA-PLG-PLA) are emulsified using a standard Silverson Laboratory mixer with ¾%-inch probe, fitted with an emulsor screen (Silverson Machines Ltd., Chesham, Bucks, UK) and solvent evaporated by the following procedure. Add 600 mg CpG—PLG-PLA or 600 mg DPLA—PLG-PLA into a screw-topped glass container fitted with a rubber or Teflon seal, and dissolve in 3.6 mL of dichloromethane. Prepare the selected tumor antigen in distilled or D.I. water to a final concentration of 1-30 mg/mL. 8 mg of polyvinyl alchohol (PVA) is added to 80 mL of boiling, distilled or D.I. water, and transferred to a screw-topped container. Add 60 mL of 8% (w/v) PVA solution to a new 150 mL beaker. Add 1 L of distilled water to a 2 L beaker, add a magnetic follower, and place on a magnetic stirrer. Using a glass pipette, aliquot 3 mL of PLG solution into a 10 mL beaker. Immerse the prove of the Silverson laboratory mixer into the PLG solution and run the mixer at full speed (8000-9000 rpm). Add the antigen solution and mix for 2.5 minutes. Immerse the probe into the PVA solution, and run the mixer at full speed. Using a glass Pasteur pipette, quickly add the PLG/antigen emulsion to the PVA and emulsify for 2.5 minutes. Add the double emulsion to the 1 L of water and stir rapidly to disperse the emulsion. Divide the suspension of nanoparticles into six 250 mL centrifuge bottles and centrifuge at 10,000×g for 20 minutes. Gently decant the liquid from the bottles without disturbing the pellets and add 10 mL of water to each bottle. Resuspend the pellets, pool, and divide equally between two bottles. Fill the bottles with water and recentrifuge at 10,000×g for 20 minutes. Wash nanoparticles twice. Resuspend and transfer the suspension to a container suitable for freeze-drying the nanoparticles. Shell freeze the suspension in solid CO₂ and freeze-dry. Transfer the freeze-dried microparticles to a glass screw-topped tube and store at −20° C. over desiccant. Micro particles containing no protein (“empty microparticles’) are made in the same way, adding water instead of antigen solution to the PLG solution in the initial emulsification stage.

### Example 14

### Addition of Tumor Cells with GM-CSF Matrix

Mouse melanoma cells (B16.F10 cells) are grown in tissue culture media. 10⁶ cells are washed in cold PBS and irradiated with 20,000 rads in the presence of complete tissue culture media using a JL Sheperd and Associates Model 109-85 Irradiator with a ⁶⁰Cobalt source to terminate cell division. After irradiation, cells are washed with complete media once then extensively with PBS and resuspended in PBS containing the matrix-GM-CSG molecules to a final concentration of 2×10⁷ cells/mL. The cell/matrix mixture is then injected intradermally in the hind fland of C57blk/6 mice. After two weeks, the mice are boosted with the same material. Mice are then challenged with 10⁶ non-irradiated wild type B16.F10 cells on the opposite flank. Tumor sizes in comparison to non-treated and irradiated wild type control vaccinations are measured over 20 days. In an experiment to examine the survival of mice using an intravenous injection, mice are previously immunized as described above then challenged using 10⁵ non-irradiated wild type cells in the tail vein. Mouse survival is measured over 30 days.

### Example 15

### Procedure for the Coupling of Antigen (e.g., Ovalbumin or KLH) to Activated (Polyaldehyde) Dextran

Dissolve the activated (polyaldehyde) dextran or buffer-exchange the activated dextran in periodate solution into 100 mM sodium phosphate, 150 mM NaCl, pH 7.2 with constant stirring to a concentration of about 10-15 mg/mL. To this mixture add 1 mg of keyhole limpet hemocyanin (KLH, molecular weight 900,000, Pierce Chemical Co.) or ovalbumin (molecular weight 43,000, Sigma Chemical Co.) (ratio of 1:1 v/v) that had been previously dialyzed into the reaction buffer (i.e. 100 mM sodium phosphate, 150 mM NaCl, pH 7.2). To this solution is added 200 μl of 1 M cyanoborohydride (Aldrich, Milwaukee, Wis.) and the mixture is allowed to react for six hours at room temperature. The remaining unreacted aldehyde groups on the dextran are blocked by adding 200 μl of 1 M Tris buffer, pH 8 and incubating the mixture an additional 2 hours at room temperature. The antigen-conjugated dextran is purified by passing this mixture through a Sephacryl S-200 or S-300.

### Example 16

### Procedure for the Coupling of GM-CSF and Antigen (e.g., Ovalbumin or KLH) to Activated (Polyaldehyde) Dextran

Dissolve the activated (polyaldehyde) dextran or buffer-exchange the activated dextran in periodate solution into 100 mM sodium phosphate, 150 mM NaCl, pH 7.2 with constant stirring to a concentration of about 10-15 mg/mL. To this mixture add 1 mg of GM-CSF and 1 mg of keyhole limpet hemocyanin (KLH, molecular weight 900,000, Pierce Chemical Co.) or ovalbumin (molecular weight 43,000, Sigma Chemical Co.) (ratio of 1:1 v/v) that had been previously dialyzed into the reaction buffer (i.e. 100 mM sodium phosphate, 150 mM NaCl, pH 7.2). To this solution is added 200 μl of 1 M cyanoborohydride (Aldrich, Milwaukee, Wis.) and the mixture is allowed to react for six hours at room temperature. The remaining unreacted aldehyde groups on the dextran are blocked by adding 200 μl of 1 M Tris buffer, pH 8 and incubating the mixture an additional 2 hours at room temperature. The antigen-conjugated dextran is purified by passing this mixture through a Sephacryl S-200 or S-300.

### Example 17

### Procedure for the Coupling of GM-CSF and Antigen (e.g., Ovalbumin or KLH) to Alginate

This method for labeling alginate utilizes existing EDC/sulfo-NHS chemistry to link the carboxylic groups on alginate to amine groups on proteins via condensation reaction. EDC acts as a water soluble reducing agent and sulfo-NHS provides a stable reactive intermediate. N-hydroxysulfosuccinimide (Sulfo-NHS) and its uncharged analog N-hydroxysuccinimide (NHS) are used to convert carboxyl groups to amine-reactive Sulfo-NHS esters. This is accomplished by mixing the Sulfo-NHS with a carboxyl containing molecule and a dehydrating agent such as the carbodiimide EDC (EDAC). EDC by itself is not particularly efficient in crosslinking because failure to react quickly with an amine will result in hydrolysis and regeneration of the carboxyl

Reaction should not be carried out in amine containing buffer (Tris, glycine, lysine, histidine). EDC and sulfo-NHS solutions should always be made up fresh Care should always be taken to prevent exposure to light

A. Alginate Labeling

Make up a solution of 1.467 wt % Alginate (High M alginate powder, 65:35, FMC biopolymer, 2%=270 cP) in PBS (no Ca2+). Aliquot 10 mls of alginate solution into 20 ml scintillation vial with small stir-bar. Add 19.5 mg hydroxysulfosuccinimide sodium salt (sulfo-NHS, Fluka a division of Sigma Aldrich, Milwaukee, Wis.), directly to alginate solution (−9 mM final conc.) Add 17.3 mg N-(3-Dimethylaminopropyl)-N′-ethylcarbodiimide hydrochloride (EDC, Fluka a division of Sigma Aldrich, Milwaukee, Wis.). React at room temperature for 2 hrs. Add 100 ul of 1.25 mg/ml (3.6 mM) GM-CSF in PBS. Solution can be made up ahead of time in PBS and stored. React overnight for approximately 18 hrs.

B. Dialysis

Load entire solution into 20 ml syringe. Rinse vial with 5 ml WFI or PBS and load into same syringe. Use syringe to load solution into dialysis cassette (30,000 MWCO, Regenerated Cellulose, Slide-a-Lyzer, Pierce). Use same syringe to remove excess air from cassette. Place Cassette in ddH₂O for 4 hrs, changing buffer once. Replace buffer with 1.0M NaCl for 4 hrs, changing buffer once. Replace buffer with ddH₂O overnight, changing buffer 5 times.

I claim:

1. A composition comprising at least three biomodulatory molecules said at least three biomodulatory molecules connected by a cross-linking agent forming a matrix wherein said matrix functions as an immuno-stimulatory adjuvant.

2. A composition according to claim 1 wherein said biomodulatory molecule is selected from the group consisting of cytokines, bacterial toxins, bacterial oligonucleotides, receptor ligands and antigen binding fragments of antibodies.

3. A composition according to claim 2 wherein said cytokine is GM-CSF, IL-2 or IL-12.

4. A composition according to claim 2 wherein said cytokines is IFN-γ or IFN-α.

5. A composition according to claim 2 wherein said cytokine is selected from the group consisting of TNF-α, TNF-β, and GM-CSF

6. A composition according to claim 2 wherein said bacterial toxin is a Staphylococcal enterotoxin or SEB.

7. A composition according to claim 2 wherein said bacterial molecule is an immunostimulatory CpG oligonucleotide motif or monophosphoryl lipid A.

8. A composition according to claim 2 wherein said antigen-binding fragments of antibodies is selected from the group consisting of anti-CD3, anti-CD40 or anti-GMCSFR.

9. A composition according to claim 2 wherein said receptor ligand is selected from the group consisting of folate, FasL and CD40.

10. The composition according to claim 1 further comprising a disease-specific antigen said disease-specific antigen able to stimulate an immune response.

11. The composition according to claim 23 wherein said disease-specific antigen is selected from the group consisting of tumor-associated antigens, infectious disease-associated antigens and viral antigen.

12. The composition of claim 24 wherein said tumor-associated antigens are selected from the group consisting of melanoma antigens, and mutants thereof, a bcr/abl breakpoint peptide, HER-2/neu and HPV.

13. The composition of claim 24 wherein said melanoma antigens are selected from the group consisting of MAGE-1, MAGE-2, MAGE-3, BAGE, GAGE-1 GAGE-2, MART-1 and tyrosinase.

14. The composition of claim 24 wherein the melanoma disease-associated antigen is gp100.

15. The composition according to claim 1 further comprising a support wherein said cross-linked biomodulatory molecules are affixed to said support.

16. The composition according to claim 29 wherein said solid support is selected from the group consisting of Dextran, polyDL lactide coglycolide, polyacrylamide, ficoll and alum.

17. A pharmaceutical composition comprising the composition according to claim 1.

18. A pharmaceutical composition comprising the composition according to claim 23.

19. A pharmaceutical composition comprising the composition according to claim 29.

20. A method of stimulating an immune response in a host by administering to said host the composition according to claim 1.

21. A method of stimulating an immune response in a host by administering to said host the composition according to claim 23.

22. A method of stimulating an immune response in a host by administering to said host the composition according to claim 29.

23. A method according to claim 37 wherein said immune response is a T cell response.

24. A method of modulating cell signal transduction in a host by administering to said host the composition according to claim 23.

25. A method of modulating cell signal transduction in a host by administering to said host the composition according to claim 29.

26. A method of treating a disease by administering to a host the composition according to claim 23.

27. A method of treating a disease by administering to a host the composition according to claim 29.


SEQUENCE LISTING (30 sequences)

SEQ ID NO: 1 - DNA, 20 bp, Unknown
  Bacterial CpG motif sequence
tccatgacgttcctgatgct

SEQ ID NO: 2 - protein, 9 aa, Homo sapien
IISAVVGIL

SEQ ID NO: 3 - protein, 9 aa, Homo sapien
KIFGSLAFL

SEQ ID NO: 4 - protein, 10 aa, Human papilloma virus
YMLDLQPETT

SEQ ID NO: 5 - protein, 20 aa, Homo sapien
PDTRPAPGSTAPPAHGVTSA

SEQ ID NO: 6 - protein, 9 aa, Homo sapien
EADPTGHSY

SEQ ID NO: 7 - protein, 9 aa, Homo sapien
SAYGEPRKL

SEQ ID NO: 8 - protein, 9 aa, Homo sapien
EVDPIGHLY

SEQ ID NO: 9 - protein, 9 aa, Homo sapien
FLWGPRALV

SEQ ID NO: 10 - protein, 9 aa, Homo sapien
AARAVFLAL

SEQ ID NO: 11 - protein, 9 aa, Homo sapien
EVDPIGHLY

SEQ ID NO: 12 - protein, 9 aa, Homo sapien
VLPDVFIRC

SEQ ID NO: 13 - protein, 9 aa, Homo sapien
AYGLDFYIL

SEQ ID NO: 14 - protein, 9 aa, Homo sapien
KTWGQYWQV

SEQ ID NO: 15 - protein, 9 aa, Homo sapien
ITDQVPFSV

SEQ ID NO: 16 - protein, 9 aa, Homo sapien
YLEPGPVTA

SEQ ID NO: 17 - protein, 10 aa, Homo sapien
LLDGTATLRL

SEQ ID NO: 18 - protein, 10 aa, Homo sapien
VLYRYGSFSV

SEQ ID NO: 19 - protein, 8 aa, Homo sapien
AAGIGLTV

SEQ ID NO: 20 - protein, 9 aa, Homo sapien
ILTVILGVL

SEQ ID NO: 21 - protein, 9 aa, Homo sapien
MSLQRQFLR

SEQ ID NO: 22 - protein, 9 aa, Homo sapien
MLLAVLYCL

SEQ ID NO: 23 - protein, 9 aa, Homo sapien
YMNGTMSQV

SEQ ID NO: 24 - protein, 9 aa, Homo sapien
SEIWRDIDF

SEQ ID NO: 25 - protein, 9 aa, Homo sapien
AFLPWHRLF

SEQ ID NO: 26 - protein, 13 aa, Homo sapien
QNILLSNAPLGPQ

SEQ ID NO: 27 - protein, 13 aa, Homo sapien
SYLQDSDPDSFQD

SEQ ID NO: 28 - protein, 9 aa, Homo sapien
SYLDSGIHF

SEQ ID NO: 29 - protein, 9 aa, Homo sapien
EEKLIVVLF

SEQ ID NO: 30 - protein, 10 aa, Homo sapien
ACDPHSGHFV